(19) United States
(12) Patent Application Publication
(10) Pub. No.: US 2005/0136025 A1
(43) Pub. Date: Jun. 23, 2005

(54) Cosmetic compositions comprising water-dispersible sulfonic polyester/polyurethane combination, and related processes

(76) Inventors: Francolse Pataut, Paris (FR); Marie-Laure Breton, Paris (FR); Jean-Luc Bremenson, Eaubonne (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/949,244

(22) Filed: Sep. 27, 2004

(30) Foreign Application Priority Data
Sep. 26, 2003 (FR) 03 11319

(51) Int. Cl. A61K 7/06; A61K 7/11

(52) U.S. Cl. 424/70.17; 424/70.11

(57) ABSTRACT

The present disclosure relates to a cosmetic composition for treating keratin materials, such as the hair, comprising, in a cosmetically acceptable aqueous medium, at least one water-dispersible sulfonic polyester and at least one polyurethane. This composition may be used for shaping and/or holding the hairstyle. The present disclosure also relates to a device comprising the composition as disclosed herein.

The present disclosure relates to a cosmetic composition for treating keratin materials, for instance the hair, to the use of this composition for shaping and/or holding the hairstyle, to an aerosol device comprising this composition and to a cosmetic treatment process using it.

In the field of styling and/or fixing the hair, known hair compositions are generally in the form of spray compositions consisting essentially of an alcoholic solution and of at least one fixing material mixed with various cosmetic adjuvants. The solution is generally packaged either in a suitable aerosol device pressurized using a propellant, or in a pump-dispenser bottle.

Numerous aerosol devices for fixing the hairstyle are known, these devices containing firstly a liquid phase (or fluid) and secondly a propellant. The function of the propellant is to provide a pressure that allows the liquid phase to be sprayed and to be applied to the hair in the form of a cloud of dispersed droplets. For ecological reasons, it is sought to reduce the amount of volatile organic compounds (or VOCs) present in the composition. To reduce the amount of VOCs and thus to obtain an aerosol device with a low content of VOCs, the organic solvents, for instance ethanol, can be partially replaced with water.

The liquid phase can, for instance, contain fixing materials, for example anionic fixing materials. However, their combination with a large amount of water can entail an increase in the viscosity of the liquid phase and the degradation of the restitution of the product, for example the production of a white spray, can be caused by the reduction in the amount of alcohols in the aerosol fluid.

Accordingly, one aspect of the present disclosure is a specific combination of at least one water-dispersible sulfonic polyester and at least one polyurethane, which makes it possible to obtain shaping and/or excellent hold of the hairstyle by giving strong and improved fixing. Another aspect of the present disclosure is when a composition comprising such a combination is packaged in aerosol form, and if the concentration of the at least one polymer is high, good restitution of the composition can be obtained since the viscosity of the liquid phase remains low. Further, the combination of the at least one water-dispersible sulfonic polyester and at least one associative or non-associative polyurethane also allows total removal of the product during shampooing.

Thus, one aspect of the present disclosure is a cosmetic composition for treating keratin materials, such as the hair, comprising the combination disclosed herein, in a cosmetically acceptable aqueous medium.

Another aspect of the present disclosure comprises the use of a composition as disclosed herein for shaping and holding the hairstyle.

Still another aspect of the present disclosure is an aerosol device comprising the composition as disclosed herein and at least one propellant.

Yet another aspect of the present disclosure is a cosmetic treatment process comprising applying the composition as disclosed herein to keratin materials.

Other subjects, characteristics, aspects and benefits of the present disclosure will emerge more clearly upon reading the description and the various examples that follow.

According to the present disclosure, the cosmetic composition for treating keratin materials, such as the hair, comprises, in a cosmetically acceptable aqueous medium, at least one water-dispersible sulfonic polyester and at least one polyurethane. This polyurethane may be associative or non-associative.

As used herein, the term “cosmetically acceptable medium” means a medium that is compatible with the hair, but that may also have a pleasant smell, appearance and feel.

As used herein, the term “water-dispersible sulfonic polyester” means any sulfonic polyester capable of forming a dispersion, i.e., a two-phase system in which the first phase is formed from finely divided particles uniformly distributed in the second phase, which is the continuous phase.

As used herein, the term “sulfonic polyester” means copolyesters obtained by polycondensation of at least one dicarboxylic acid, which may be for instance aromatic, and for example, different from terephthalic acid or an ester thereof or one of its salts, of at least one diol, and optionally an other diol and of at least one sulfoaryldicarboxylic difunctional compound substituted on the aromatic nucleus with a group —SO₃M wherein M is chosen from a hydrogen atom and metal ions, such as Na⁺, Li⁺ or K⁺. The water-dispersible sulfonic polyester can be obtained for example, from isophthalic acid optionally in ester form and sulfoisophtalic acid optionally in salt form as sole dicarboxylic acids present, diethylene glycol and at least one polyurethane. Diols used in the sulfonic polyester synthesis according to the present disclosure, include, for example, diethylene glycol, dipropylene glycol, tripropylene glycol, polyethylene glycol, butane-(1,4)-diol, and 1,4-cyclohexanemethanol.

The water-dispersible sulfonic polyesters generally have a weight-average molecular mass ranging from 1,000 to 60,000, such as from 4,000 to 20,000, as determined by gel permeation chromatography (or GPC). The glass transition temperature of the sulfonic polyesters as disclosed herein ranges from 10° C. to 100° C., for instance from 25 to 60° C. They are described in greater detail in U.S. Pat. Nos. 3,734,874; 3,779,993; 4,119,680; 4,300,580; 4,973,656; 5,660,816; 5,662,893; and 5,674,479.

For further example, the sulfonic polyesters that can be used in according to the present disclosure can comprise at least some units derived from sole isophthalic acid as dicarboxylic acid, sulfoaryldicarboxylic acid salt and diethylene glycol, for instance, the sulfonic polyesters used in the according to the present disclosure can be obtained from sole isophthalic acid as dicarboxylic acid, the sodium salt of sulfoisophthalic acid, diethylene glycol and 1,4-cyclohexanemethanol.

Non-limiting examples of sulfonic polyesters that may be used as disclosed herein include, for instance, those known under the INCI name diglycol/CHDM/isophthalates/SIP, and sold under the trade names Eastman AQ® by the company Eastman Chemical, such as the product sold under the trade name Eastman AQ 48®.

For the purposes of the present disclosure, the term “non-associative polyurethane” means polycondensates comprising at least one polyurethane block and not comprising, in their structure, any terminal or pendent alkyl or alkenyl chains comprising more than 10 carbon atoms. These are described, for example, in European Patent Nos. EP 0 751 162, EP 0 637 600, and EP 0 648 485, and French Patent No. FR 2,743,297 from L'Oreal, as well as EP 0 656 021 or WO 94/03510 from the company BASF, and EP 0 619 111 from the company National Starch.

The non-associative polyurethanes used in accordance with the present disclosure may be soluble in the cosmetically acceptable aqueous medium, for instance after neutralization with an organic or mineral base, or alternatively they may form a dispersion in this medium. The dispersion can, in this case, comprise at least 0.05% surfactant for dispersing the non-associative polyurethane and holding it in dispersion.

According to the present disclosure, any type of surfactant may be used in the dispersion, including, for example, a nonionic surfactant. The mean particle size of the non-associative polyurethane in the dispersion can range, for example, from 0.1 to 1 micrometer.

By way of non-limiting example, the non-associative polyurethane may be formed by an arrangement of blocks, this arrangement being obtained, for example, from:
(1) at least one compound which comprises at least two active hydrogen atoms per molecule;
(2) at least one diol, a mixture of diols comprising acid functional groups, and salts thereof; and
(3) at least one di- or polyisocyanate.

For instance, the compounds of type (1) can be chosen from diols, diamines, polyesterols, polyetherols, and mixtures thereof. For further instance, the compounds of type (1) may be chosen from linear polyethylene glycols and linear polypropylene glycols, such as those obtained by reacting ethylene oxide or propylene oxide with water or diethylene glycol or dipropylene glycol in the presence of sodium hydroxide as catalyst. These polyalkylene glycols can have a molecular mass ranging from 600 to 20,000.

Other non-limiting examples of organic compounds that may be used include those comprising mercapto, amino, carboxyl and hydroxyl groups. Among these, non-limiting mention may be made of, for example, polyhydroxylated compounds such as polyetherdiols, polyesterdiols, polyacetaldiols, polyamidediols, polyesterpolyamidediols, poly(alkylene ether)diols, polythioetherdiols and polycarbonatediols.

The polyetherdiols that may be used include, for example, the products of condensation of ethylene oxide, propylene oxide or tetrahydrofuran, the grafted or block products of copolymerization or of condensation thereof, such as mixtures of condensates of ethylene oxide and of propylene oxide, and the products of polymerization of olefins, at high pressure, with alkylene oxide condensates. Suitable polyetherdiols are prepared, for example, by condensation of alkylene oxides and of polyhydric alcohols, such as ethylene glycol, 1,2-propylene glycol and 1,4-butanediol.

The polyesterdiols, polyesteramides and polyamidediols can be, for example, saturated, and can be obtained, for example, from the reaction of saturated or unsaturated polycarboxylic acids with polyhydric alcohols, diamines or polyamines. Adipic acid, succinic acid, phthalic acid, terephthalic acid and maleic acid may be used, for example, to prepare these compounds. Polyhydric alcohols that are suitable for preparing polyesters include, for example, ethylene glycol, 1,2-propylene glycol, 1,4-butanediol, neopentyl glycol and hexanediol. Amino alcohols, for example ethanolamine, may also be used. Diamines that are suitable for preparing the polyesteramides are ethylenediamine and hexamethylenediamine.

Suitable polyacetals may be prepared, for example, from 1,4-butanediol or hexanediol and formaldehyde. Suitable polythioethers may be prepared, for example, by condensation reaction between thioglycols alone or in combination with other glycols such as ethylene glycol, 1,2-propylene glycol or with other polyhydroxylated compounds. Polyhydroxylated compounds already comprising urethane groups, natural polyols, which may be further modified, for example castor oil and carbohydrates, may also be used.

For example, the compound of type (1) can be a polyesterol, such as polyesterdiol formed by the reaction of at least one (di)polyol (1ₐ) and at least one acid (1_{b}). The (di)polyol (1ₐ) can be chosen, for example, from the group comprising neopentyl glycol, 1,4-butanediol, hexanediol, ethylene glycol, diethylene glycol, propylene glycol, butylene glycol, neopentyl glycol and (di)polyethylene glycol. The acid (1_{b})can be chosen, for example, from the group comprising phthalic acid, isophthalic acid, adipic acid and (poly)lactic acid.

A hydroxycarboxylic acid such as dimethylolpropanoic acid (DMPA) or a 2,2-hydroxymethylcarboxylic acid may be used, for example, as a compound of type (2). In general, compounds of type (2) are useful as coupling blocks. Compounds of type (2) that may used as disclosed herein include those comprising at least one α,α-dihydroxylated carboxylic acid.

Non-limiting examples of the compounds of type (2) that may be used in accordance with the present disclosure include, for example, those chosen from 2,2-di(hydroxymethyl)acetic acid, 2,2-dihydroxymethylpropionic acid, 2,2-dihydroxymethylbutyric acid and 2,2-dihydroxymethylpentanoic acid.

The di- or polyisocyanate of type (3) may be chosen for example, from the group comprising hexamethylene diisocyanate, isophorone diisocyanate (IDPI), toluylene diisocyanate, 4,4′-diphenylmethane diisocyanate (DPMD), 4,4′-dicyclohexylmethane diisocyanate (DCMD), methylenebis(p-phenyl diisocyanate), methylenebis(4-cyclohexyl isocyanate), toluene diisocyanates, 1,5-napthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanates, 2,2′-dichloro-4,4′-diisocyanatodiphenylmethane, 2,4-dibromo-1,5-diisocyanatonaphthalene, 1,4-butane diisocyanate, 1,6-hexane diisocyanate and 1,4-cyclohexane diisocyanate.

The non-associative polyurethane may be formed using an additional compound of type (4) that generally serves to lengthen its chain. These compounds of type (4) may be chosen, for example, from saturated and unsaturated glycols such as ethylene glycol, diethylene glycol, neopentyl glycol and triethylene glycol; amino alcohols such as ethanolamine, propanolamine and butanolamine; heterocyclic, aromatic, cycloaliphatic and aliphatic primary amines; diamines; carboxylic acids such as aliphatic, aromatic and heterocyclic carboxylic acids, for instance oxalic acid, succinic acid, glutaric acid, adipic acid, sebacic acid and terephthalic acid; and aminocarboxylic acids. In one embodiment of the present disclosure, the compounds of type (4) are aliphatic diols.

The non-associative polyurethanes used according to the present disclosure may also be formed from additional compounds of type (5) having a silicone skeleton, such as polysiloxanes, polyalkylsiloxanes or polyarylsiloxanes, for instance, polyethylsiloxanes, polymethylsiloxanes and polyphenylsiloxanes, optionally comprising hydrocarbon-based chains grafted onto the silicon atoms.

The non-associative polyurethanes that may be used can comprise, for example, a base repeating unit chosen from those of formula (I):
—O—B—O—CO—NH—R—NH—CO— (I)
wherein:
B is chosen from divalent C₁ to C₃₀ hydrocarbon-based groups, which are optionally substituted with a group comprising at least one carboxylic acid functional group and/or at least one sulfonic acid functional group, the carboxylic acid and/or sulfonic acid functional groups being in free form or partially or totally neutralized with a mineral or organic base, and
R is a divalent group chosen from C₁ to C₂₀ aliphatic hydrocarbon-based groups, C₃ to C₂₀ cycloaliphatic groups and C₆ to C₂₀ aromatic groups, for instance C₁ to C₂₀ alkylene, C₆ to C₂₀ arylene or C₃ to C₂₀ cycloalkylene groups, or combinations thereof, these groups being substituted or unsubstituted.

For example, the group R may be chosen from groups of the formulae: 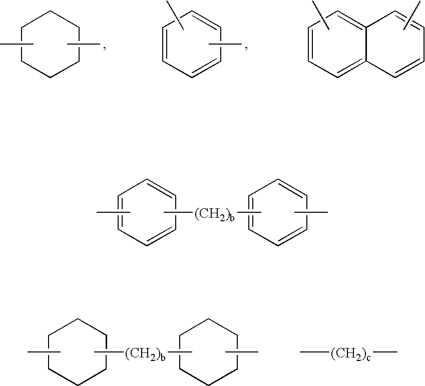
wherein b is an integer ranging from 0 to 3, and c is an integer ranging from 1 to 20, such as from 2 to 12.

For instance, the group R may be chosen from hexamethylene, 4,4′-biphenylenemethane, 2,4- and/or 2,6-tolylene, 1,5-naphthylene, p-phenylene and methylene-4,4-bis(cyclohexyl) groups and the divalent group derived from isophorone.

The non-associative polyurethane used according to the present disclosure may also comprise for example, at least one polysiloxane block, its base repeating unit being chosen from, for example, those of formula (II):
—O—P—O—CO—NH—R—NH—CO— (II)
wherein:
P is a polysiloxane segment, and
R is a divalent group chosen from C₁ to C₂₀ aliphatic hydrocarbon-based groups, C₃ to C₂₀ cycloaliphatic groups and C₆ to C₂₀ aromatic groups, for instance C₁ to C₂₀ alkylene, C₆ to C₂₀ arylene or C₃ to C₂₀ cycloalkylene groups, or combinations thereof, these groups being substituted or unsubstituted.

For instance, the polysiloxane segment P can be chosen from polysiloxane segments of formula (III): 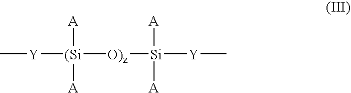
wherein:
A, which may be identical or different, are chosen from monovalent C₁ to C₂₀ hydrocarbon-based groups that are free or substantially free of ethylenic unsaturation, and aromatic groups,
Y is chosen from divalent hydrocarbon-based groups, and
z is an integer chosen such that the weight-average molecular mass of the polysiloxane segment ranges from 300 to 10,000.

For example, the divalent group Y may be chosen from the alkylene groups of formula —(CH₂)ₐ—, in which “a” is an integer ranging from 1 to 10.

The groups A may be chosen from C₁ to C₈ alkyl groups, for instance, methyl, ethyl, propyl, isopropyl, butyl, pentyl, hexyl and octyl groups; C₃ to C₈ cycloalkyl groups, such as cyclohexyl groups; C₆ to C₁₀ aryl groups, for example phenyl; C₇ to C₁₀ arylalkyl groups, for instance benzyl, phenylethyl, tolyl and xylyl groups.

Non-limiting examples of non-associative polyurethanes that may be mentioned include the dimethylolpropionic acid/isophorone diisocyanate/neopentyl glycol/polyesterdiols copolymer (also known under the name polyurethane-1, INCI name) sold under the brand name Luviset® Pur by the company BASF, and the dimethylolpropionic acid/isophorone diisocyanate/neopentyl glycol/polyesterdiols/silicone diamine copolymer (also known under the name polyurethane-6, INCI name) sold under the brand name Luviset® Si Pur A by the company BASF.

The non-associative polyurethanes may be present in the composition according to the present disclosure in an amount, for example, ranging from 0.05% to 20% by weight, for instance from 0.1% to 10% by weight, such as from 1% to 8% by weight, relative to the total weight of the cosmetic composition for treating keratin materials.

As used herein, the term “associative polyurethane” means a polyurethane having at least one terminal or pendent alkyl chain comprising at least 10 carbon atoms. This type of polymer is capable of interacting with itself or with particular compounds, such as surfactants, to produce thickening of the medium.

The associative polyurethanes used according to the present disclosure are chosen from cationic, anionic and nonionic associative polyurethanes. In one embodiment of the present disclosure, they are chosen from anionic and nonionic associative polyurethanes.

Among the anionic associative polyurethanes that may be used as disclosed herein, non-limiting mention may be made of acrylic terpolymers that are soluble or swellable in alkalis. Such acrylic terpolymers comprise:
a) from 20% to 70% by weight, such as from 25% to 55% by weight, of a carboxylic acid comprising α, β-monoethylenic unsaturation;
b) from 20% to 80% by weight, such as from 30% to 65% by weight, of a non-surfactant monomer comprising monoethylenic unsaturation, which is different from a), and c) from 0.5% to 60% by weight, such as from 10% to 50% by weight, of a nonionic urethane monomer which is the product of reaction of a monohydric nonionic surfactant with a monoisocyanate comprising monoethylenic unsaturation.

The carboxylic acid comprising α, β-monoethylenic unsaturation can be chosen from many acids, for example acrylic acid, methacrylic acid, itaconic acid and maleic acid. In one embodiment of the present disclosure, methacrylic acid is used. A large proportion of acid is used in order to give a polymer structure which dissolves and gives a thickening effect by reaction with an alkaline compound such as sodium hydroxide, alkanolamines, aminomethylpropanol and aminomethylpropanediol.

The terpolymer also comprises a large proportion, indicated above, of a monomer comprising monoethylenic unsaturation which has no surfactant properties. For example, the monomers that can be used include those which give polymers that are water-insoluble when they are homopolymerized, for instance C₁-C₄ alkyl acrylates and methacrylates such as methyl acrylate, ethyl acrylate and butyl acrylate, or corresponding methacrylates. In one embodiment of the present disclosures, the monomers are methyl and ethyl (meth)acrylates. Other non-limiting examples of monomers which can be used include styrene, vinyltoluene, vinyl acetate, acrylonitrile and vinylidene chloride. For example, non-reactive monomers may be used, such monomers being those in which the single ethylenic group is the only group which is reactive under the polymerization conditions. However, monomers that comprise groups that are reactive under the action of heat can be used in certain situations, such as hydroxyethyl acrylate.

The monohydric nonionic surfactants used to obtain the nonionic urethane monomer are known and are for example, alkoxylated hydrophobic compounds comprising an alkylene oxide forming the hydrophilic part of the molecule. The hydrophobic compounds comprise an aliphatic alcohol or an alkylphenol in which a carbon chain comprising at least six carbon atoms constitutes the hydrophobic part of the surfactant.

For example, the preferred monohydric nonionic surfactants can be chosen from those of formula: 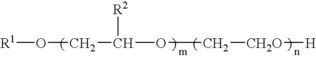
wherein R¹ is chosen from C₆-C₃₀ alkyl and C₈-C₃₀ aralkyl groups, R² is chosen from C₁-C₄ alkyl groups, n is an average number ranging from 5 to 150 and m is an average number ranging from 0 to 50, with the condition that n is at least as large as m and that the sum (n+m) ranges from 5 to 150.

Among the C₆-C₃₀ alkyl groups that may be used, non-limiting mention may be made of dodecyl and C₁₈-C₂₆ alkyl radicals. Among the aralkyl groups that may be used, non-limiting mention may be made of (C₈-C₁₃)alkylphenyl groups. In one embodiment of the present disclosure, R² is a methyl group.

The monoisocyanate comprising monoethylenic unsaturation, which is used to form the nonionic urethane monomer can be chosen from a wide variety of compounds. A compound comprising any copolymerizable unsaturation such as acrylic or methacrylic unsaturation can be used. An allylic unsaturation imparted by allyl alcohol can also be used. For further example, the monoethylenic monoisocyanates may be α,α-dimethyl-m-isopropenyl-benzylisocyanate and methylstyrene-isopropylisocyanate.

The acrylic terpolymer defined above is obtained by aqueous emulsion copolymerization of the components a), b) and c) which is known and described in patent application EP-A-0 173 109.

Non-limiting examples of anionic associative polyurethanes that may be used according to the present disclosure, include, for example, copolymers of methacrylic or acrylic acid comprising at least one C₁ to C₃₀ alkyl (meth)acrylate unit and a urethane unit substituted with a fatty chain. Non-limiting mention may also be made, for example, of the methacrylic acid/methyl methacrylate/methylstyrene-isopropyl isocyanate/behenyl alcohol polyethoxylated copolymer (comprising 40 ethoxy units) sold under the brand name Viscophobe® DB 1000 by the company Union Carbide.

The nonionic associative polyurethanes used according to the present disclosure include, for example, polyurethane polyethers comprising in their chain both hydrophilic blocks, for instance, of polyoxyethylenated nature, and hydrophobic blocks that may be aliphatic sequences alone and/or cycloaliphatic and/or aromatic sequences.

For example, the polyurethane polyethers can comprise at least two hydrocarbon-based lipophilic chains comprising from 6 to 30 carbon atoms, separated by a hydrophilic block, the hydrocarbon-based chains possibly being pendent chains, or chains at the end of the hydrophilic block. For instance, it is possible for at least one pendent chain to be included. In addition, the polymer may comprise a hydrocarbon-based chain at one end or at both ends of a hydrophilic block.

The polyurethane polyethers may be multiblock, such as in triblock form. Hydrophobic blocks may be at each end of the chain (for example: triblock copolymer with a hydrophilic central block) or distributed both at the ends and in the chain (for example: multiblock copolymer). These same polymers may also be graft polymers or starburst polymers.

The nonionic fatty-chain polyurethane polyethers may be triblock copolymers in which the hydrophilic block is a polyoxyethylenated chain comprising from 50 to 1,000 oxyethylene groups. The nonionic polyurethane polyethers comprise a urethane linkage between the hydrophilic blocks, whence arises the name. Further, also included among the nonionic fatty-chain polyurethane polyethers are those in which the hydrophilic blocks are linked to the lipophilic blocks via other chemical bonds.

As further non-limiting examples of nonionic fatty-chain polyurethane polyethers that may be used according to the present disclosure, it is also possible to use Rheolate 205 comprising a urea function, sold by the company Rheox, or Rheolate 208, 204 or 212, and also Acrysol® RM 184. Non-limiting mention may also be made of the product Elfacos T210 comprising a C₁₂ to C₁₄ alkyl chain, and the product Elfacos® T212 comprising a C₁₈ alkyl chain, from Akzo. The product DW 1206B from Rohm & Haas comprising a C₂₀ alkyl chain and a urethane linkage, sold at a solids content of 20% in water, may also be used.

It is also possible to use solutions or dispersions of these polymers, for instance in water or in aqueous-alcoholic medium. Non-limiting examples of such polymers that may be mentioned include Rheolate® 255, Rheolate® 278 and Rheolate® 244 sold by the company Rheox. The products DW 1206F and DW 1206J sold by the company Rohm & Haas may also be used.

The polyurethane polyethers that may be used according to the present disclosure include, for example, those described in the article by G. Fonnum, J. Bakke and F k. Hansen—Colloid Polym. Sci 271, 380-389 (1993).

Further non-limiting examples of nonionic associative polyurethanes that may be mentioned include polyurethane polyethers that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate. Such polyurethane polyethers are sold for example, by the company Rohm & Haas under the names Aculyn® 46 and Aculyn® 44. Aculyn® 46 is a polycondensate of polyethylene glycol comprising 150 or 180 mol of ethylene oxide, of stearyl alcohol and of methylenebis(4-cyclohexylisocyanate) (SMDI), at 15% by weight in a matrix of maltodextrin (4%) and water (81%); Aculyn® 44 is a polycondensate of polyethylene glycol comprising 150 or 180 mol of ethylene oxide, of decyl alcohol and of methylenebis(4-cyclohexylisocyanate) (SMDI), at 35% by weight in a mixture of propylene glycol (39%) and water (26%).

The anionic or nonionic associative polyurethanes may be present in the composition according to the present disclosure, for example, in an amount ranging from 0.05% to 20% by weight, such as from 0.1% to 10% by weight, and for instance from 1% to 8% by weight, relative to the total weight of the cosmetic composition for treating keratin materials.

The at least one polyurethane used according to the present disclosure is soluble in the medium.

The at least one sulfonic polyester is present in the composition according to the present disclosure in a amount ranging from 0.1% to 40% by weight, such as from 1% to 30% by weight, for instance, from 5% to 25% by weight, relative to the total weight of the cosmetic composition for treating keratin materials.

The cosmetically acceptable aqueous medium may consist solely of water or comprise a mixture of water and of at least one cosmetically acceptable solvent, such as a C₁-C₄ lower alcohol, for instance ethanol, isopropanol, tert-butanol or n-butanol; alkylene polyols, for instance propylene glycol; polyol ethers; and mixtures thereof. In one embodiment of the present disclosure, the additional solvent is ethanol.

The amount of water present in the compositions of the present disclosure can range from 15% to 95%, such as from 20% to 90%, and for instance, from 25% to 80% by weight, relative to the total weight of the composition.

The composition according to the present disclosure may also comprise at least one standard additive that is known in the art, such as other cationic, amphoteric, zwitterionic, anionic or nonionic polymers different from those described above; thickeners; plasticizers; nacreous agents; opacifiers; UV-screening agents; sugars; fragrances; mineral, plant and/or synthetic oils; fatty acid esters; dyes; volatile or non-volatile, organomodified or non-organomodified, cyclic or acyclic, branched or unbranched silicones; mineral or organic, natural or synthetic particles; preserving agents and pH stabilizers.

A person skilled in the art will take care to select the optional additives and the amount thereof such that they do not harm the properties of the compositions of the present disclosure. These additives are present in the composition according to the present disclosure in an amount ranging from 0 to 20% by weight, relative to the total weight of the composition.

The cosmetic compositions for treating keratin materials in accordance with the present disclosure may be in the form of a foam, a gel, a spray or a lacquer, and may be used in rinse-out or leave-in application.

The compositions in accordance with the present disclosure may be used as products for shaping and/or holding the hairstyle, haircare compositions, shampoos, hair conditioning compositions, such as compositions for giving the hair softness, or hair makeup compositions. In one embodiment of the present disclosure, they are compositions for shaping and/or holding the hairstyle.

The compositions according to the present disclosure can be packaged in a common cosmetic aerosol device. In such a case, at least one propellant that is known in the art may be used, such as hydrocarbon-based gases, for instance C₃ to C₅ alkanes, for example propane, n-butane or isobutane; fluorinated gases; nitrogen, air and carbon dioxide; dimethyl ether; and mixtures thereof. In one embodiment of the present disclosure, the at least one propellant is chosen from dimethyl ether, hydrocarbon-based gases, and mixtures thereof.

The at least one propellant can be present in an amount ranging from 5% to 80% by weight, for instance from 5% to 60% by weight, such as from 5% to 40% by weight, relative to the total weight of the cosmetic composition for treating keratin materials.

The present disclosure also relates to a cosmetic process for treating the hair, for example a styling process, which comprises applying an effective amount of a composition as disclosed herein to the hair, and in rinsing it out or leaving it in after an optional leave-in time.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The example that follows is given as a non-limiting illustration of the present disclosure.

### EXAMPLE

A styling composition was prepared in the form of an aerosol spray containing 55% by weight of volatile organic compounds, from the following ingredients. The amounts are indicated as percentages by weight:

| Sulfonic polyester: Eastman AQ 48 ® sold by Eastman | 3% AM* |
|---|---|
| Chemical | |
| Non-associative fixing polyurethane: Luviset ® Si Pur | 4% AM* |
| sold by BASF | |
| Ethanol | 20% |
| Dimethyl ether | 35% |
| Water | 38% |

*AM = active material

This styling product was sprayed onto the hair and the hairstyle was shaped. Good hairstyle hold was obtained.

1. A cosmetic composition for treating keratin materials, comprising, in a cosmetically acceptable aqueous medium, at least one water-dispersible sulfonic polyester and at least one polyurethane,
wherein the at least one water-dispersible sulfonic polyester is obtained from dicarboxylic acids chosen from isophthalic acid optionally in ester form and sulfoisophtalic acid optionally in salt form; and diethylene glycol.

2. The cosmetic composition for treating keratin materials according to claim 1, wherein the dicarboxylic acids are chosen from isophtalic acid or an ester thereof, and at least one salt of sulfoisophtalic acid.

3. The cosmetic composition for treating keratin materials according to claim 2, wherein the dicarboxylic acids are chosen from isophtalic acid and the sodium salt of sulfoisophtalic acid.

4. The cosmetic composition for treating keratin materials according to claim 1, wherein the at least one water-dispersible sulfonic polyester is obtained from isophthalic acid, the sodium salt of sulfoisophthalic acid diethylene glycol and at least one other diol.

5. The cosmetic composition for treating keratin materials according to claim 4, wherein the water-dispersible sulfonic polyester is obtained from isophthalic acid, the sodium salt of sulfoisophthalic acid, diethylene glycol and 1,4-cyclohexanemethanol.

6. A cosmetic composition for treating keratin materials, comprising, in a cosmetically acceptable aqueous medium, at least one water-dispersible sulfonic polyester and at least one polyurethane soluble in the medium.

7. The cosmetic composition for treating keratin materials according to claim 6, wherein the at least one water-dispersible sulfonic polyester is a polycondensate of at least one dicarboxylic acid or an ester thereof, of at least one diol and of at least one sulfoaryldicarboxylic difunctional compound substituted on the aromatic nucleus with a group —SO₃M, wherein M is chosen from a hydrogen atom and metal ions.

8. The cosmetic composition for treating keratin materials according to claim 7 wherein the metal ions are chosen from such as Na+, Li+ and K+.

9. The cosmetic composition for treating keratin materials according to claim 7, wherein the dicarboxylic acid is aromatic.

10. The cosmetic composition for treating keratin materials according to claim 1, wherein the at least one polyurethane is associative.

11. The cosmetic composition for treating keratin materials according to claim 6, wherein the at least one polyurethane is associative.

12. The cosmetic composition for treating keratin materials according to claim 1, wherein the at least one polyurethane is non-associative.

13. The cosmetic composition for treating keratin materials according to claim 6, wherein the at least one polyurethane is non-associative.

14. The cosmetic composition for treating keratin materials according to claim 12, wherein the at least one non-associative polyurethane comprises a repeating base unit of formula (I):
—O—B—O—CO—NH—R—NH—CO— (I)
wherein:
B is a divalent group chosen from C₁ to C₃₀ hydrocarbon-based groups, optionally substituted with a group comprising at least one carboxylic acid functional group and/or at least one sulfonic acid functional group, the carboxylic acid and/or sulfonic acid functional groups being in free form or partially or totally neutralized with a mineral or organic base, and
R is a divalent group chosen from C₁ to C₂₀ aliphatic hydrocarbon-based groups, C₃ to C₂₀ cycloaliphatic groups, C₆ to C₂₀ aromatic groups, and combinations thereof, these groups being substituted or unsubstituted.

15. The cosmetic composition for treating keratin materials according to claim 13, wherein the at least one non-associative polyurethane comprises a repeating base unit of formula (I):
—O—B—O—CO—NH—R—NH—CO— (I)
wherein:
B is a divalent group chosen from C₁ to C₃₀ hydrocarbon-based groups, optionally substituted with a group comprising at least one carboxylic acid functional group and/or at least one sulfonic acid functional group, the carboxylic acid and/or sulfonic acid functional groups being in free form or partially or totally neutralized with a mineral or organic base, and
R is a divalent group chosen from C₁ to C₂₀ aliphatic hydrocarbon-based groups, C₃ to C₂₀ cycloaliphatic groups, C₆ to C₂₀ aromatic groups, and combinations thereof, these groups being substituted or unsubstituted.

16. The cosmetic composition for treating keratin materials according to claim 14, wherein the at least one non-associative polyurethane is a dimethylolpropionic acid/isophorone diisocyanate/neopentyl glycol/polyesterdiol copolymer.

17. The cosmetic composition for treating keratin materials according to claim 16, wherein the at least one non-associative polyurethane is a dimethylolpropionic acid/isophorone diisocyanate/neopentyl glycol/polyesterdiol copolymer.

18. The cosmetic composition for treating keratin materials according to claim 12, wherein the at least one non-associative polyurethane also comprises a repeating base unit of formula (II):
—O—P—O—CO—NH—R—NH—CO— (II)
wherein:
P is a polysiloxane segment, and
R is a divalent group chosen from C₁ to C₂₀ aliphatic hydrocarbon-based groups, C₃ to C₂₀ cycloaliphatic groups, C₆ to C₂₀ aromatic groups, and combinations thereof, these groups being substituted or unsubstituted.

19. The cosmetic composition for treating keratin materials according to claim 13, wherein the at least one non-associative polyurethane also comprises a repeating base unit of formula (II):
—O—P—O—CO—NH—R—NH—CO— (II)
wherein:
P is a polysiloxane segment, and
R is a divalent group chosen from C₁ to C₂₀ aliphatic hydrocarbon-based groups, C₃ to C₂₀ cycloaliphatic groups, C₆ to C₂₀ aromatic groups, and combinations thereof, these groups being substituted or unsubstituted.

20. The cosmetic composition for treating keratin materials according to claim 18, wherein the at least one non-associative polyurethane is a dimethylolpropionic acid/isophorone diisocyanate/neopentyl glycol/polyesterdiol/silicone diamine copolymer.

21. The cosmetic composition for treating keratin materials according to claim 19, wherein the at least one non-associative polyurethane is a dimethylolpropionic acid/isophorone diisocyanate/neopentyl glycol/polyesterdiol/silicone diamine copolymer.

22. The cosmetic composition for treating keratin materials according to claim 12, wherein the at least one non-associative polyurethane is present in an amount ranging from 0.05% to 20% by weight, relative to the total weight of the composition.

23. The cosmetic composition for treating keratin materials according to claim 22, wherein the at least one non-associative polyurethane is present in an amount ranging from 0.10% to 10% by weight, relative to the total weight of the composition.

24. The cosmetic composition for treating keratin materials according to claim 13, wherein the at least one non-associative polyurethane is present in an amount ranging from 0.05% to 20% by weight, relative to the total weight of the composition.

25. The cosmetic composition for treating keratin materials according to claim 24, wherein the at least one non-associative polyurethane is present in an amount ranging from 0.10% to 10% by weight, relative to the total weight of the composition.

26. The cosmetic composition for treating keratin materials according to claim 10, wherein the at least one associative polyurethane is chosen from cationic, anionic and nonionic associative polyurethanes.

27. The cosmetic composition for treating keratin materials according to claim 11, wherein the at least one associative polyurethane is chosen from cationic, anionic and nonionic associative polyurethanes.

28. The cosmetic composition for treating keratin materials according to claim 26, wherein the at least one anionic associative polyurethane is an acrylic terpolymer comprising:
a)- from 20% to 70% by weight, of a carboxylic acid comprising α, β-monoethylenic unsaturation;
from 20% to 80% by weight, of a non-surfactant monomer comprising monoethylenic unsaturation, which is different from a), and
c) from 0.5% to 60% by weight, of a nonionic urethane monomer which is the product of reaction of a monohydric nonionic surfactant with a monoisocyanate comprising monoethylenic unsaturation.

29. The cosmetic composition for treating keratin materials according to claim 27, wherein the at least one anionic associative polyurethane is an acrylic terpolymer comprising:
a) from 20% to 70% by weight, of a carboxylic acid comprising α, β-monoethylenic unsaturation;
b) from 20% to 80% by weight, of a non-surfactant monomer comprising monoethylenic unsaturation, which is different from a), and
c) from 0.5% to 60% by weight, of a nonionic urethane monomer which is the product of reaction of a monohydric nonionic surfactant with a monoisocyanate comprising monoethylenic unsaturation.

30. The cosmetic composition for treating keratin materials according to claim 28, wherein the at least one anionic associative polyurethane is a methacrylic acid/methyl methacrylate/methylstyrene-isopropyl isocyanate/behenyl alcohol polyethoxylated copolymer comprising 40 ethoxy units.

31. The cosmetic composition for treating keratin materials according to claim 29, wherein the at least one anionic associative polyurethane is a methacrylic acid/methyl methacrylate/methylstyrene-isopropyl isocyanate/behenyl alcohol polyethoxylated copolymer comprising 40 ethoxy units.

32. The cosmetic composition for treating keratin materials according to claim 26, wherein the at least one nonionic associative polyurethane is a polyurethane polyether that may be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

33. The cosmetic composition for treating keratin materials according to claim 27, wherein the at least one nonionic associative polyurethane is a polyurethane polyether that may be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

34. The cosmetic composition for treating keratin materials according to claim 32, wherein the at least one nonionic associative polyurethane is chosen from polycondensates of polyethylene glycol comprising 150 or 180 mol of ethylene oxide, stearyl alcohol and methylenebis(4-cyclohexyl isocyanate), and polycondensates of polyethylene glycol comprising 150 or 180 mol of ethylene oxide, decyl alcohol and methylenebis(4-cyclohexyl isocyanate).

35. The cosmetic composition for treating keratin materials according to claim 33, wherein the at least one nonionic associative polyurethane is chosen from polycondensates of polyethylene glycol comprising 150 or 180 mol of ethylene oxide, stearyl alcohol and methylenebis(4-cyclohexyl isocyanate), and polycondensates of polyethylene glycol comprising 150 or 180 mol of ethylene oxide, decyl alcohol and methylenebis(4-cyclohexyl isocyanate).

36. The cosmetic composition for treating keratin materials according to claim 26, wherein the at least one associative polyurethane is present in a total amount ranging from 0.05% to 20% by weight, relative to the total weight of the composition.

37. The cosmetic composition for treating keratin materials according to claim 36, wherein the at least one associative polyurethane is present in a total amount ranging from 0.1% to 10% by weight, relative to the total weight of the composition.

38. The cosmetic composition for treating keratin materials according to claim 27, wherein the at least one associative polyurethane is present in a total amount ranging from 0.05% to 20% by weight, relative to the total weight of the composition.

39. The cosmetic composition for treating keratin materials according to claim 38, wherein the at least one associative polyurethane is present in a total amount ranging from 0.1% to 10% by weight, relative to the total weight of the composition.

40. The cosmetic composition for treating keratin materials according to claim 1, wherein the at least one sulfonic polyester is present in the composition in a total amount ranging from 0.1% to 40% by weight, relative to the total weight of the cosmetic composition for treating keratin materials.

41. The cosmetic composition for treating keratin materials according to claim 40, wherein the at least one sulfonic polyester is present in the composition in a total amount ranging from 1% to 30% by weight, relative to the total weight of the cosmetic composition for treating keratin materials.

42. The cosmetic composition for treating keratin materials according to claim 41, wherein the at least one sulfonic polyester is present in the composition in a total amount ranging from 5% to 25% by weight, relative to the total weight of the cosmetic composition for treating keratin materials.

43. The cosmetic composition for treating keratin materials according to claim 6, wherein the at least one sulfonic polyester is present in the composition in a total amount ranging from 0.1% to 40% by weight, relative to the total weight of the cosmetic composition for treating keratin materials.

44. The cosmetic composition for treating keratin materials according to claim 43, wherein the at least one sulfonic polyester is present in the composition in a total amount ranging from 1% to 30% by weight, relative to the total weight of the cosmetic composition for treating keratin materials.

45. The cosmetic composition for treating keratin materials according to claim 44, wherein the at least one sulfonic polyester is present in the composition in a total amount ranging from 5% to 25% by weight, relative to the total weight of the cosmetic composition for treating keratin materials.

46. The cosmetic composition for treating keratin materials according to claim 1, wherein the cosmetically acceptable aqueous medium consists of water or comprises a mixture of water and at least one cosmetically acceptable solvent.

47. The cosmetic composition for treating keratin materials according to claim 6, wherein the cosmetically acceptable aqueous medium consists of water or comprises a mixture of water and at least one cosmetically acceptable solvent.

48. The cosmetic composition for treating keratin materials according to claim 46, wherein the at least one cosmetically acceptable solvent is chosen from C₁-C₄ lower alcohols, alkylene polyols, polyol ethers, and mixtures thereof.

49. The cosmetic composition for treating keratin materials according to claim 47, wherein the at least one cosmetically acceptable solvent is chosen from C₁-C₄ lower alcohols, alkylene polyols, polyol ethers, and mixtures thereof.

50. The cosmetic composition for treating keratin materials according to claim 48, wherein the at least one cosmetically acceptable solvent is chosen from ethanol, isopropanol, tert-butanol, n-butanol and propylene glycol.

51. The cosmetic composition for treating keratin materials according to claim 49, wherein the at least one cosmetically acceptable solvent is chosen from ethanol, isopropanol, tert-butanol, n-butanol and propylene glycol.

52. The cosmetic composition for treating keratin materials according to claim 50, wherein the cosmetically acceptable solvent is ethanol.

53. The cosmetic composition for treating keratin materials according to claim 51, wherein the cosmetically acceptable solvent is ethanol.

54. The cosmetic composition for treating keratin materials according to claim 1, wherein water is present in an amount ranging from 15% to 95% by weight, relative to the total weight of the composition.

55. The cosmetic composition for treating keratin materials according to claim 54, wherein water is present in an amount ranging from 20% to 90% by weight, relative to the total weight of the composition.

56. The cosmetic composition for treating keratin materials according to claim 6, wherein water is present in an amount ranging from 15% to 95% by weight, relative to the total weight of the composition.

57. The cosmetic composition for treating keratin materials according to claim 56, wherein water is present in an amount ranging from 20% to 90% by weight, relative to the total weight of the composition.

58. The cosmetic composition for treating keratin materials according to claim 1, further comprising at least one additive chosen from other cationic, amphoteric, zwitterionic, anionic or nonionic polymers different from those comprised in the composition; thickeners; plasticizers; nacreous agents; opacifiers; UV-screening agents; sugars; fragrances; mineral, plant and/or synthetic oils; fatty acid esters; dyes; volatile or non-volatile, organomodified or non-organomodified, cyclic or acyclic, branched or unbranched silicones; mineral or organic, natural or synthetic particles; preserving agents and pH stabilizers.

59. The cosmetic composition for treating keratin materials according to claim 6, further comprising at least one additive chosen from other cationic, amphoteric, zwitterionic, anionic or nonionic polymers different from those comprised in the composition; thickeners; plasticizers; nacreous agents; opacifiers; UV-screening agents; sugars; fragrances; mineral, plant and/or synthetic oils; fatty acid esters; dyes; volatile or non-volatile, organomodified or non-organomodified, cyclic or acyclic, branched or unbranched silicones; mineral or organic, natural or synthetic particles; preserving agents and pH stabilizers.

60. The cosmetic composition for treating keratin materials according to claim 1, wherein it is in the form of a foam, a gel, a spray or a lacquer.

61. The cosmetic composition for treating keratin materials according to claim 6, wherein it is in the form of a foam, a gel, a spray or a lacquer.

62. An aerosol device comprising a cosmetic composition for treating keratin materials comprising
a cosmetic composition for treating keratin materials, comprising, in a cosmetically acceptable aqueous medium, at least one water-dispersible sulfonic polyester, at least one polyurethane, and at least one propellant,
wherein the at least one water-dispersible sulfonic polyester is obtained from dicarboxylic acids chosen from isophthalic acid optionally in ester form and sulfoisophtalic acid optionally in salt form; and diethylene glycol.

63. The aerosol device according to claim 62, wherein the at least one propellant is chosen from hydrocarbon-based gases, fluorinated gases, nitrogen, air, carbon dioxide, dimethyl ether, and mixtures thereof.

64. The aerosol device according to claim 63, wherein the at least one propellant is chosen from dimethyl ether, hydrocarbon-based gases, and mixtures thereof.

65. The aerosol device according to claim 62, wherein the at least one propellant is present in an amount ranging from 5% to 80% by weight, relative to the total weight of the composition.

66. The aerosol device according to claim 65, wherein the at least one propellant is present in an amount ranging from 5% to 60% by weight, relative to the total weight of the composition.

67. A cosmetic process for treating the hair, comprising applying to the hair a cosmetic treatment composition comprising, in a cosmetically acceptable aqueous medium, at least one water-dispersible sulfonic polyester and at least one polyurethane soluble in the medium.

68. A cosmetic process for treating the hair, comprising applying to the hair a cosmetic treatment composition comprising, in a cosmetically acceptable aqueous medium, at least one water-dispersible sulfonic polyester, at least one polyurethane, and at least one propellant,
wherein the at least one water-dispersible sulfonic polyester is obtained from dicarboxylic acids chosen from isophthalic acid optionally in ester form and sulfoisophtalic acid optionally in salt form; and diethylene glycol.
